(19) United States
(12) Patent Application Publication
(10) Pub. No.: US 2020/0176096 A1
(43) Pub. Date: Jun. 4, 2020

(54) MEDICAL INFORMATION DEVICE, MEDICAL INFORMATION SYSTEM, AND METHOD FOR MEDICAL INFORMATION PROCESSING

(71) Applicant: Konica Minolta Healthcare Americas, Inc., Wayne, NJ (US)

(72) Inventor: Hiroyuki Kubota, Wayne, NJ (US)

(73) Assignee: Konica Minolta Healthcare Americas, Inc., Wayne, NJ (US)

(21) Appl. No.: 16/206,367

(22) Filed: Nov. 30, 2018

(51) Int. Cl. G16H 15/00; G16H 30/40; G16H 10/60

(52) U.S. Cl. CPC G16H 15/00; G16H 10/60; G16H 30/40

(57) ABSTRACT

A medical information device includes a storage and a processor. The storage stores a medical report including a plurality of items, wherein each of the items includes an information content and reference information. The information content of at least one of the items of the medical report is a medical image. The reference information includes: a medical-code system value; and a medical-code value that corresponds to the information content and is defined in a medical-code system identified by the medical-code system value. The processor determines whether the information content will be displayed on a display client device based on the medical-code value; and upon determining that the information content will be displayed, transmits the information content and the reference information to the display client device.

### BACKGROUND

Electronic medical records, including medical images and other medical data play a crucial role in the diagnosis of patients. Healthcare facilities (e.g., hospitals) have realized the benefits of electronically storing medical records. The digitalization of medical images and other data not only enables users to easily access the medical images and medical data, but also enables the images and data to be easily shared between multiple healthcare facilities.

In the healthcare industry, the use of a system known as a Picture Archiving and Communications System (“PACS”) is becoming increasingly popular for convenient storage and access of medical images and reports. Generally, a PACS comprises a multitude of devices working cooperatively to digitally capture, store, manage, distribute, and display medical images generated by various imaging modalities, such as computed tomography (CT), magnetic resonance imaging (MRI), position emission tomography (PET), ultrasound, X-ray, etc. PACS allows various healthcare facilities to share all types of images and reports captured or created internally or externally.

Usually, these medical reports containing the diagnosis of the medical image are created by using a template selected from a plurality of templates. The reporter creates the medical report by filling in the blanks of the template. In many cases, the healthcare facilities have their own templates to create their own medical reports. However, the templates are sometimes revised or removed from the storage after the medical reports have been created. Or, sometimes the reports are moved or copied from a device with templates to a medical information device without templates. In these cases, the medical reports cannot be displayed or be utilized through the medical information device storing the medical reports because the medical information device does not also store the templates.

### SUMMARY

One or more embodiments of this invention provide a medical information device, a medical information system, and a method for medical information processing that creates medical reports that can be displayed even if a template used at the time of creating the medical report no longer exists or is no longer stored.

One or more embodiments provides a medical information device including: a storage that stores a medical report including a plurality of items, wherein each of the items includes: an information content, wherein the information content of at least one of the items of the medical report is a medical image; and reference information including: a medical-code system value; and a medical-code value that corresponds to the information content and is defined in a medical-code system identified by the medical-code system value; and a processor that: determines whether the information content will be displayed on a display client device based on the medical-code value; and upon determining that the information content will be displayed, transmits the information content and the reference information to the display client device.

The plurality of items may be stored in an order to be displayed in the medical report; and the information content is stored after the reference information in each of the items.

The medical report may further include a template identifier that identifies a template having a layout for displaying the information content of each of the items in the medical report; and the processor: may further determine, when the medical report is requested from the display client device, whether the template identified by the template identifier exists, and upon determining that the template exists, transmit the template instead of the reference information.

The information content, when the information content is the medical image, may be stored as a link to the medical image stored in a medical image server.

The processor may further store: a specific type of the medical imaging device; a specific area of the human body part that is captured as the medical image by the medical imaging device.

The reference information may further include: a caption to be displayed together with the information content; and an information type of the information content.

The processor may transmit the medical report to the display client device; and upon receiving the medical report, the display client device may adjust a size of a display area displaying the information content of each of the items of the medical report based on the reference information.

The reference information may further include information composed of at least one word in a text format.

One or more embodiments provides a medical information system including: an editing client device that transmits an information content of a medical report; the medical information device according to claim 1, wherein the medical information device receives the information content from the editing client device and transmits the information content and reference information that corresponds to the information content; a display client device that receives the information content and the reference information and displays the medical report according to the reference information.

The display client device, when the display client device receives the information content and the reference information, may adjust the size of the display area displaying the information content based on the reference information.

The display client device, when the display client device receives the information content and the reference information, may determine whether to display the information content based on the reference information.

One or more embodiments provides a method for medical information processing, including: storing a medical report including a plurality of items, wherein each of the items includes: an information content, wherein the information content of at least one of the items of the medical report is a medical image; and reference information including: a medical-code system value; and a medical-code value that corresponds to the information content and is defined in a medical-code system identified by the medical-code system value; and deciding whether the information content is to be displayed based on the medical-code value; and transmitting the information content and the reference information to display at a display device when the processor determines the information content is to be displayed.

### BRIEF DESCRIPTION OF THE DRAWINGS

FIG. 1 shows a diagram of the medical information system according to one or more embodiments.

FIG. 2 shows a hardware diagram of the medical information device according to one or more embodiments.

FIG. 3 shows a diagram indicating storage regions of the storage of the medical information device according to one or more embodiments.

FIG. 4 shows an exemplary structure of the report region of the storage.

FIG. 5 shows an exemplary structure of a report content.

FIG. 6 shows an exemplary structure of an item in the medical report.

FIG. 7 shows a sample code of the medical report.

FIG. 8 shows a sequence diagram of the medical information system according to one or more embodiments.

FIG. 9 shows a flow chart for the transmission process of the medical information device.

FIG. 10 shows an example of a screen image of the medical report displayed on the display device.

FIG. 11 shows a diagram indicating another usage of the medical information device without utilizing any networks according to one or more embodiments.

### DETAILED DESCRIPTION

Specific embodiments of the invention will now be described in detail with reference to the accompanying figures. Like elements in the various figures are denoted by like reference numerals for consistency.

In the following detailed description of embodiments of the invention, numerous specific details are set forth in order to provide a more thorough understanding of the invention. However, it will be apparent to one of ordinary skill in the art that the invention may be practiced without these specific details. In other instances, well-known features have not been described in detail to avoid unnecessarily complicating the description.

[Medical Information System]

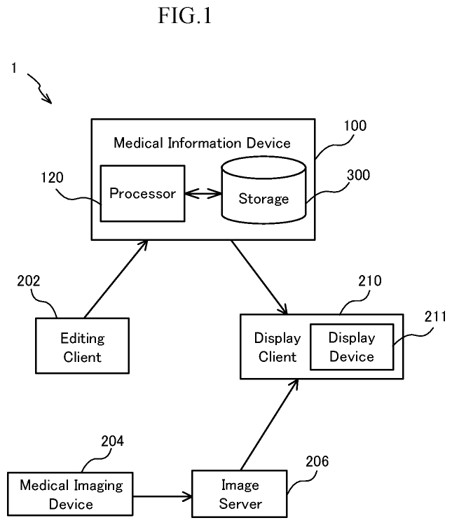

FIG. 1 shows a diagram of the medical information system 1 according to one or more embodiments. As shown in FIG. 1, the medical information system 1 includes a medical information device 100 and a display client device 210. The medical information system 1 may further include an editing client device 202, an image server 206, and a medical imaging device 204.

The editing client device 202 is operated by a user to edit a medical report 310. When the medical report 310 is edited, the editing client device sends an information content 350 of the medical report 310 to the medical information device 100. The medical information device 100 receives the information content 350 from the editing client device 202 and transmits the information content 350 and the reference information 340 that corresponds to the information content 350 to the display client device 210. The display client device 210 receives the information content 350 and the reference information 340 and displays the medical report 310 on the display device 211, according to the reference information 340. The display device 211 can be a LCD (Liquid Crystal Display) display, an OLED (Organic Light Emitting Diode) display, a CRT (Cathode Ray Tube) display, or projection type display, etc.

The medical imaging device 204 may include devices that can capture images of computed tomography (CT), magnetic resonance imaging (MRI), position emission tomography (PET), ultrasound, X-ray, etc. The medical imaging device 204 may send their captured images to the image server 206 to store them. The medical images stored in the image server 206 may be identified by unique identification code 350B. The medical images stored on the image server 206 can be referred or retrieved as a part of the medical report on the display client device 210 by using the unique identification code 350B.

[Medical Information Device]

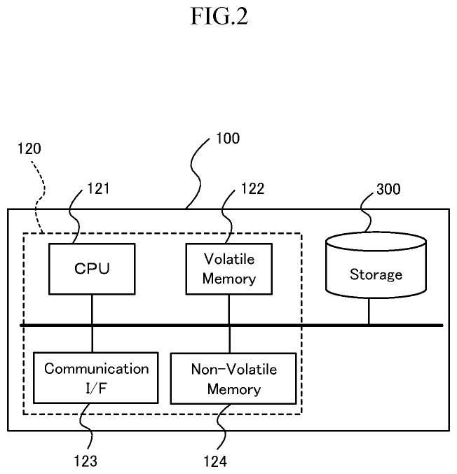

FIG. 2 shows a hardware diagram of the medical information device 100 according to one or more embodiments. As shown in FIG. 2, the medical information device 100 is an information processing device including a storage 300, a CPU (central processing unit) 121, a volatile memory 122, a non-volatile memory 124, and a communication interface 123. The storage 300 may be non-volatile memory such as hard disks or flash memories, etc., and may store medical reports 310. The volatile memory 122 can include RAM (random access memory) or cache memory, etc. The non-volatile memory 124 can include ROM (read only memory), flash memory or hard disk, etc. The communication interface 123 connects to a network to communicate with other devices, such as the editing client device 202 and the display client device 210. In the following description, the CPU 121, the volatile memory 122, non-volatile memory, and communication interface 123 are collectively referenced as processor 120. However, it is also possible that the processor 120 includes only the CPU 121, or includes CPU 121 and any one or more of the volatile memory 122, the non-volatile memory, and the communication interface 123, etc. The processor 120 stores the medical reports 310 to the storage 300 and transmits the medical report 310 to display on the display device 211 of the display client device 210.

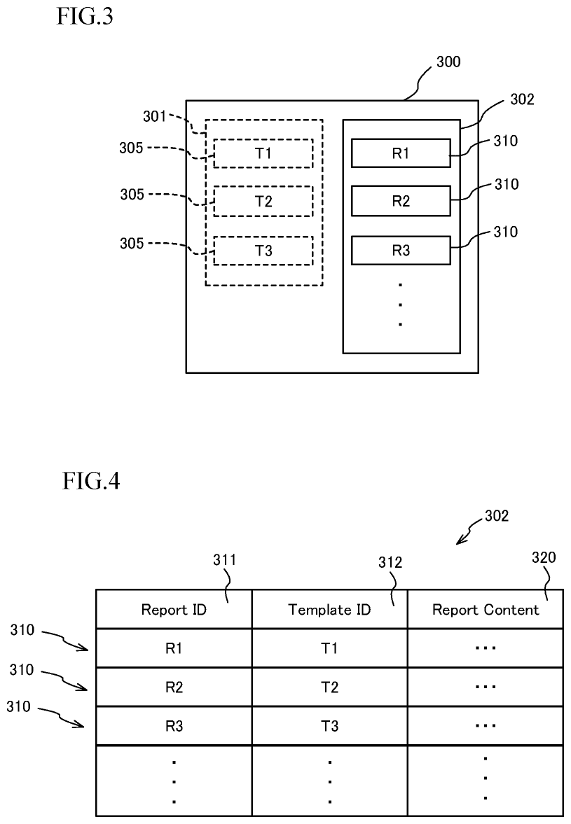

FIG. 3 shows a diagram indicating storage regions of the storage 300 of the medical information device 100 according to one or more embodiments. The storage 300 includes a report region 302 and a template region 301. The report region 302 can store the plurality of the medical reports 310. The template region 301 can store the plurality of templates 305. Each template 305 has a layout for displaying the information content 350 of each of the items 330 (described later) in the medical report 310. Because the template region 301 or the templates 305 may not exist in the storage 300 when the medical report 310 is requested to be displayed, they are indicated by broken lines in FIG. 3. These cases sometimes occur when the templates 305 may be revised or removed from the storage 300 after the medical report 310 has been edited or when the reports 310 may be moved or copied from another device to the medical information device 100 without having the template region 301.

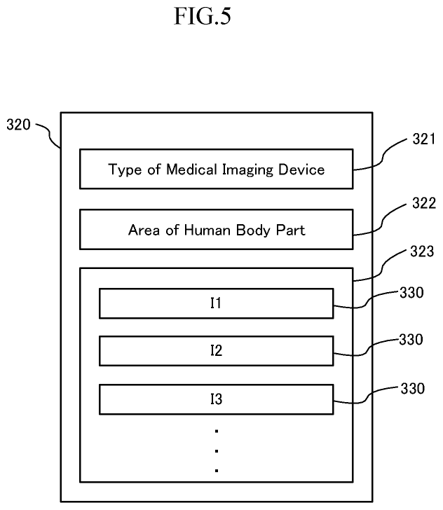

FIG. 4 shows an exemplary structure of the report region 302 of the storage 300. Each medical report 310 is identified by a report ID (identifier) 311. Each medical report 310 may further include a template ID 312 that identifies a template 305, and a report content 320.

FIG. 5 shows an exemplary structure of the report content 320. The report content 320 may identify a specific type of a medical imaging device 321 and a specific area of the human body of a human body part 322 that is captured as a medical image 350A by the medical imaging device 204. The specific type of the medical imaging device 321 and the specific area of the human body part 322 are stored by the processor 120. The report content 320 further includes an item region 323 that includes a plurality of items 330, each of which includes an information content 350 displayed on the display device 211. The plurality of items 330 stored in the report content 320 is arranged in an order to be displayed in the medical report 310.

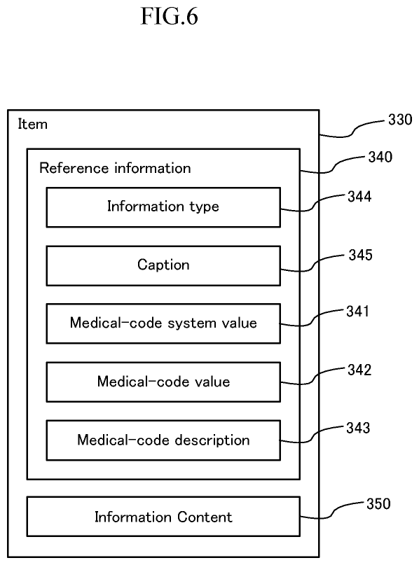

FIG. 6 shows an exemplary structure of the item 330 in the medical report 310. The item 330 includes the reference information 340 and the information content 350. The reference information 340 includes an information type 344 of the information content 350. The information type 344 may be related to how the information content 350 is displayed. That is, the information type 344 may be stored as at least one word in a text format such as “numeric,” “image,” “text,” “radio button,” or “combo box,” etc. The reference information 340 may further include a caption 345, a medical-code system value 341, a medical-code value 342, and a medical-code description 343. The caption 345 is stored as at least one word in a text format and displayed together with the information content 350 when the medical report 310 is displayed. The medical-code value 342 corresponds to the information content 350, and is defined in medical-code system identified by the medical-code system value 341. The medical-code system may be a system defined generally in public or also may be a system privately defined by a user or healthcare facilities. The medical-code description 343 can be text data explaining what is indicated by the medical-code value 342 and can be displayed together with the information content 350. The information content 350 may be stored in the region after the region that stores the reference information 340 in each of the items 330.

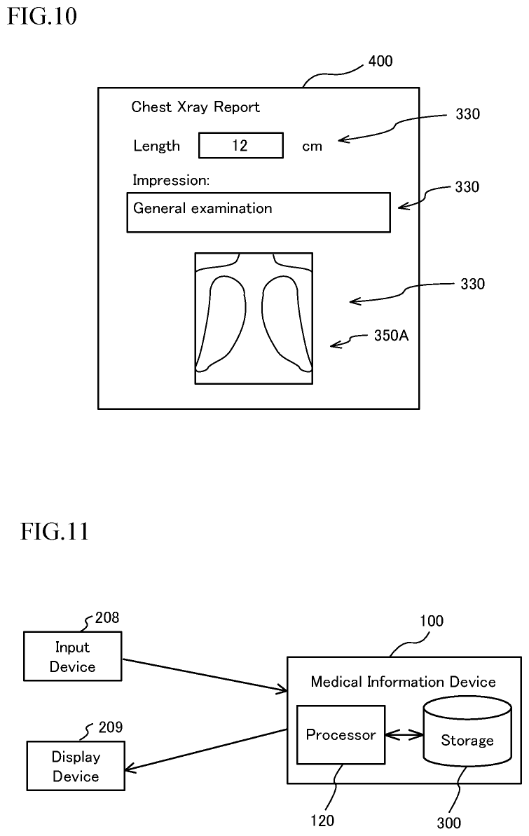

The information content 350 of at least one of the items 330 of the medical report 310 is a medical image 350A (as shown in FIG. 10). In this case, the information content 350 can be stored as a link to the medical image 350A, stored in the image server 206, by using the unique identification code 350B.

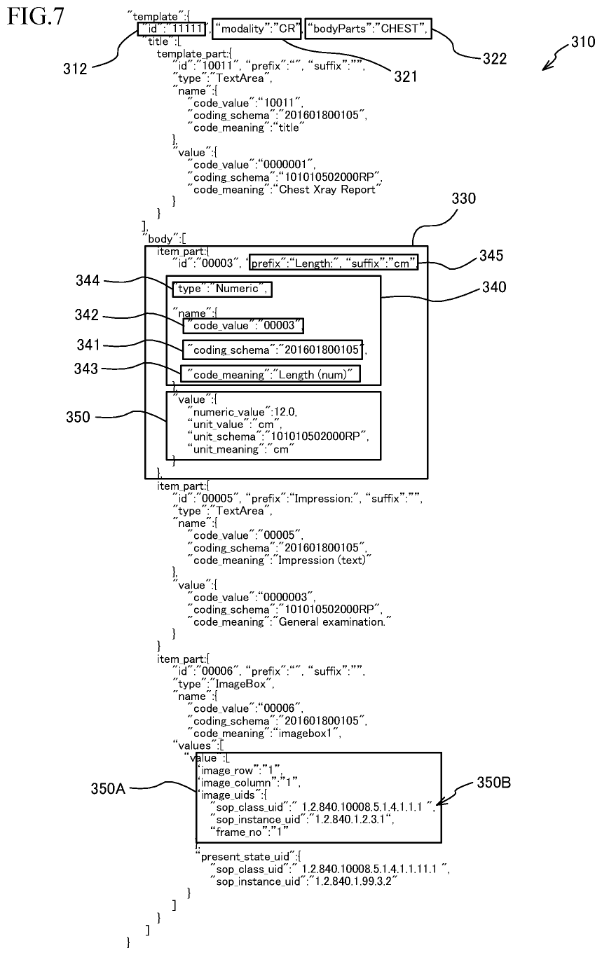

FIG. 7 shows a sample code of the medical report 310. The sample code is written by using JSON (JavaScript® Object Notation) format, although any format to store to the storage 300 can be employed. According to the sample code, the template ID 312 identifies a template that is employed at the time of editing the medical report 310. The specific type of the medical imaging device 321 indicates that the medical report 310 is based on the medical image captured by “CR” (Computed Radiography). The specific area of the human body part 322 indicates the medical image is an image of “CHEST.” The specific area could be any area of the human body like a head, arm, hand, stomach, leg, foot, etc. In the reference information 340 of the item 330, the information type 344 of the information content 350 is indicated as “numeric.” The medical-code value 342 of the medical-code system indicates “length” as shown in the medical-code description 343. In the information content 350, the value of the “numeric” is indicated as “12.” The caption 345 indicates that “Length” is displayed before “12,” and “cm” is displayed after “12” when the medical report 310 is displayed. The medical image 350A is also included as an information content of one of the items 330. The medical image 350A may be identified by the unique identification code 350B.

Because the template ID 312 is stored together with the information content 350, the medical information device 100 can recognize which template is suitable for displaying the medical report 310 by referring to the template ID 312. Because the specific type of the medical imaging device 321 and the specific area of the human body part 322 are stored together with the information content 380, the medical information device 100 can cause the display client device 210 to collectively display all relevant stored data so that the user may recognize what kind of image and which part of the body the report is based on. Because the information type 344 of the information content 350 is stored together with the information content 350, the medical information device 100 causes the display client device 210 to display the information content 350 by utilizing an appropriate display style such as a text box, image box etc. depending on the information type 344. Because the medical-code value 342 of the medical-code system is stored together with the information content 350, the medical information device 100 also causes the display client device 210 to display the information content 350 by utilizing an appropriate display style such as the size or the resolution of the image, the additional buttons for switching images etc. depending on the medical-code value 342, and the medical information device 100 can determine whether the information content 250 is to be displayed, based on the medical-code value 342. Because the caption 345 is stored together with the information content 350, the caption can be displayed together with the information content 350. Because the medical image 350A may be stored by the link of a public accessible server in the medical report 310, the display client device 210 can display the medical image 350A anywhere the display client device is located. Because some of the information stored in the reference information such as the caption 345 or the information type 344 may be stored as at least one word in a text format, viewers can recognize the content of the medical report 310 by not only the screen on the display but also by the source code of the medical report 310.

[Operation of Medical Information System]

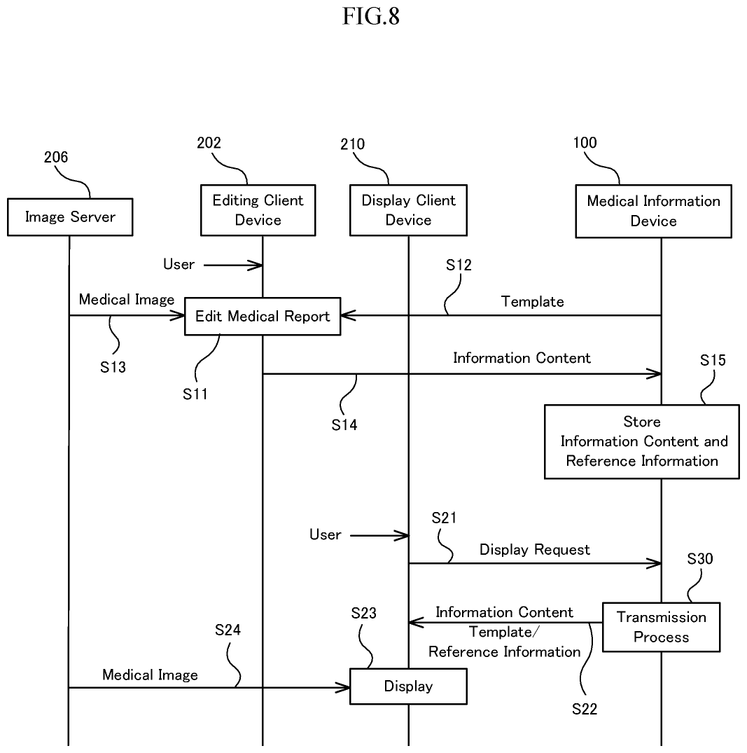

FIG. 8 shows a sequence diagram of the medical information system 1 according to one or more embodiments. As shown in FIG. 8, at first, a user edits a medical report 310 on the editing client device 202 (Step S11). When the user edits the medical report 310, the editing client device retrieves a template 305 from the medical information device 100 (Step S12) and retrieves a medical image 350A from the image server 206 (Step S13) to edit the medical report 310. After editing the medical report 310, the information content 350 of each of the items 330 is transmitted to the medical information device 100 (Step S14). When the medical information device 100 receives the information content 350 of each of the items 330, the processor 120 of the medical information device 100 stores the information content 350 and the reference information 340, which constitute the medical report 310 (Step S15).

When the display client device 210 requests to display a medical report 310 to the medical information device 100 according to the operation of a user (Step S21), the medical information device executes the transmission process S30 described later. During the execution of the transmission process S30, the information content 350, with the template 305 or the reference information 340, is transmitted to the display client device 210. The display client device 210 displays the information content 350 of each of the items according to the template 305 or the reference information 340 of each of the items on the display device 211 (Step S23). That is, the display client device 210 displays the medical report 310 on the display device 211. When the display client device 210 displays the medical report 310, the display client device retrieves the medical image 350A from the image server 206. In this sequence diagram, the display client device 210 is depicted as a different device from the editing client device 202; however, the display client device 210 may be the same device as the editing client device 202.

[Operation of Medical Information Device]

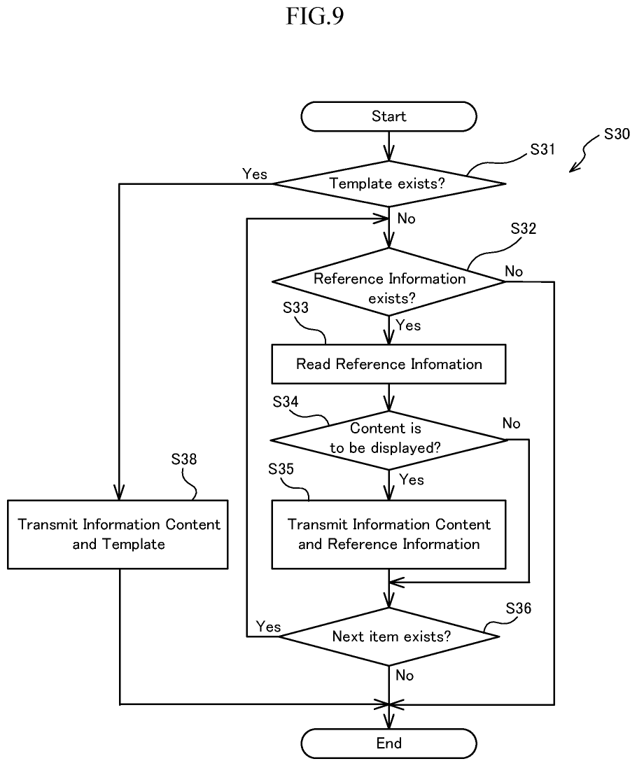

FIG. 9 shows a flow chart in accordance with one or more embodiments for the transmission process S30 of the medical information device 100 when the medical information device 100 is requested to display the medical report 310 by the display client device 210. When the medical report 310 is requested to be displayed, the processor 120 determines whether the template 305 identified by the template ID 312 exists (Step S31). When the processor 120 determines that the template 305 exists (Step S31: Yes), the processor 120 transmits the template 305 together with the information content 350 of each of the items 330 to display on the display device 211 by using the template 305 (Step S38) and ends the transmission process S30. When the processor 120 determines that the template 305 does not exist (Step S31: No), the processor 120 further determines whether the reference information 340 exists (Step S32). When the processor 120 determines that the reference information 340 does not exist (Step S32: No), the processor 120 ends the transmission process S30. When the processor 120 determines that the reference information 340 exists (Step S32: Yes), the processor 120 reads the reference information 340 of the first item 330 of the medical report 310 (Step S33), and determines whether the information content 350 will be displayed (Step S34). When the processor 120 determines that the information content 350 will not be displayed (Step S34: No), the process proceeds to Step S36. When the processor 120 determines that the information content 350 will be displayed (Step S34: Yes), the processor 120 transmits the reference information 340, together with the information content 350 of each of the items 330, to display on the display device 211 by using the reference information 340 (Step S35). At Step S36, the processor 120 determines whether the next item 330 exists in the medical report 310 requested to be displayed. When the processor 120 determines that the next item 330 exists, the processor 120 returns to process Step S32, relating to the next item 330. When the processor 120 determines that the next item 330 does not exist, the transmission process S30 is ended.

Thus, the medical information device 100 can cause the display client device 210 to display the medical report 310 by using the template 305 when the template 305 exists, and it can cause the display client device 210 to display the medical report 310 by using the reference information 340 when the template 305 does not exist. Therefore, the medical information device 100 can cause the display client device 210 to display the medical report 310 even if the template 305 does not exist. Further, the processor 120 can select the information content 350 to be transmitted based on the medical-code value 342. Thus, the medical information device 100 can cause the display client device 210 to display the information content depending on the kind of the display client devices 210 or the credentials of the user of the display client devices 210, e.g. patients, doctors, or researchers

In the transmission process S30 shown in FIG. 9, the processor 120 determines whether the information content 350 to be transmitted. However, the display client device 210, instead of the processor 120, may determine whether the information content 350 is to be displayed. That is, when the display client device 210 receives the information content 350 and the reference information 340, the display client device 210 may determine whether to display the information content 350 based on the reference information 340.

FIG. 10 shows an example of a screen image 400 of the medical report 310 displayed on the display device 211 when the template 305 does not exist. As shown in FIG. 10, the information content 350 of each of the items 330 is shown according to the reference information 340 of each of the items 330. One of the information contents 350 is the medical image 350A. Other information contents 350 are created by the user based on the medical image 350A. When the display client device 210 receives the information content 350 and the reference information 340, the display client device 210 can adjust the size of the display area displaying the information content 350 of each of the items 330 based on the display size of the display device 211 and the reference information 340, e.g. the information type 344, or the medical-code value 342 of the medical-code system, etc. The plurality of items 330 are displayed in an order as stored in the report content 320.

FIG. 11 shows a diagram indicating another configuration of the medical information device 100 without utilizing any networks. In the medical information system 1 shown in FIG. 1, the processor 120 of the medical information device 100 transmits the medical report 310, which includes the reference information 340 and the information content 350, to the display client device 210. However, the processor 120 of the medical information device 100 may transmit the medical report 310 to the display device 209 directly, without utilizing any communication networks between the medical information device 100 and the display client device 210. Also, the medical reports 310 can be edited directly at the medical information device 100 itself without connecting to the editing client device 202 through a communication network. The input device 208—such as a keyboard, a mouse, or a touch panel—could be connected to the medical information device to edit medical reports 310. The same advantageous effects could be obtained even in the configuration shown in FIG. 11.

One or more embodiments of the present invention provide the following technological improvements in computer functionality. One or more embodiments of the present invention cause the medical information device to create medical reports that are displayed even if a template used at the time of creating the medical report no longer exists or is no longer stored, that causes the medical reports to be viewable in any computers. Thus, the medical reports can be circulated easier and easily be shared, that helps e.g. doctors who need the information of the medical reports. Further, the report structure of the items, each of which is constituted by the combination of the information content and the reference information, allows the computer to determine whether the item will be displayed, what style the item to be displayed etc. Thus, for example, this function can change the display style of the information content depending on the information content such as CR image for chest as a human body part or MRI image for head as a human body part etc., or depending on the person requesting to view the report such as a patient, researcher, doctor etc.

Although the disclosure has been described with respect to only a limited number of embodiments, those skilled in the art, having benefit of this disclosure, will appreciate that various other embodiments may be devised without departing from the scope. Accordingly, the scope of the invention should be limited only by the attached claims.

1. A medical information device comprising:
a storage that stores a medical report including a plurality of items, wherein each of the items comprises:
an information content, wherein the information content of at least one of the items of the medical report is a medical image; and
reference information comprising:
a medical-code system value; and
a medical-code value that corresponds to the information content and is defined in a medical-code system identified by the medical-code system value; and
a processor that:
determines whether the information content will be displayed on a display client device based on the medical-code value; and
upon determining that the information content will be displayed, transmits the information content and the reference information to the display client device.

2. The medical information device according to claim 1, wherein:
the plurality of items are stored in an order to be displayed in the medical report; and
the information content is stored after the reference information in each of the items.

3. The medical information device according to claim 1, wherein:
the medical report further comprises a template identifier that identifies a template having a layout for displaying the information content of each of the items in the medical report; and
the processor:
further determines, when the medical report is requested from the display client device, whether the template identified by the template identifier exists, and
upon determining that the template exists, transmits the template instead of the reference information.

4. The medical information device according to claim 1, wherein when the information content is the medical image, the information content is stored as a link to the medical image stored in a medical image server.

5. The medical information device according to claim 4, wherein the processor further stores:
a specific type of the medical imaging device;
a specific area of the human body part that is captured as the medical image by the medical imaging device.

6. The medical information device according to claim 1, wherein the reference information further comprises:
a caption to be displayed together with the information content; and
an information type of the information content.

7. The medical information device according to claim 1, wherein:
the processor transmits the medical report to the display client device; and
upon receiving the medical report, the display client device adjusts a size of a display area displaying the information content of each of the items of the medical report based on the reference information.

8. The medical information device according to claim 1, wherein the reference information further comprises information composed of at least one word in a text format.

9. A medical information system comprising:
an editing client device that transmits an information content of a medical report;
the medical information device according to claim 1, wherein the medical information device receives the information content from the editing client device and transmits the information content and reference information that corresponds to the information content;
a display client device that receives the information content and the reference information and displays the medical report according to the reference information.

10. The medical information system according to claim 9, wherein when the display client device receives the information content and the reference information, the display client device adjusts the size of the display area displaying the information content based on the reference information.

11. The medical information system according to claim 9, wherein when the display client device receives the information content and the reference information, the display client device determines whether to display the information content based on the reference information.

12. A method for medical information processing, comprising:
storing a medical report comprising a plurality of items, wherein each of the items comprises:
an information content, wherein the information content of at least one of the items of the medical report is a medical image; and
reference information comprising:
a medical-code system value; and
a medical-code value that corresponds to the information content and is defined in a medical-code system identified by the medical-code system value; and
deciding whether the information content is to be displayed based on the medical-code value; and
transmitting the information content and the reference information to display at a display device when the processor determines the information content is to be displayed.
